(19) United States
(12) Patent Application Publication
(10) Pub. No.: US 2015/0306325 A1
(43) Pub. Date: Oct. 29, 2015

(54) SYSTEM FOR PROVIDING PRESSURE PULSES TO THE AIRWAY OF A SUBJECT

(71) Applicant: Koninklijke Philips N.V., Eindhoven (NL)

(72) Inventor: Manuel LAURA LAPOINT, Pittsburgh, PA (US)

(21) Appl. No.: 14/437,377

(22) PCT Filed: Nov. 6, 2013
(86) PCT No.: PCT/IB2013/059947

§ 371 Date: Apr. 21, 2015

Related U.S. Application Data
(60) Provisional application No. 61/724,432, filed on Nov. 9, 2012.

(51) Int. Cl. A61M 16/00; A61M 16/20

(52) U.S. Cl. CPC A61M 16/0069; A61M 16/0003; A61M 16/208; A61M 2205/3334; A61M 2205/50

(57) ABSTRACT

A pressure generation system for a pressure support system (10), the pressure generation system comprising: a pressure generator (14) for delivery of breathable gas to the airway of a subject (12); one or more sensors (18) sensing one or more parameters of the pressurized flow of breathable gas; and one or more processors (20) comprising: a respiratory event detection module (54) configured to detect airway obstruction, a pulse parameter module (56), the pulse configured to open the airway of the subject; and a control module (58) configured to control the pressure generator to generate, responsive to detection of airway obstruction, the pulse of breathable gas for delivery to the airway of the subject in accordance with the obtained pulse parameters.

The present disclosure pertains to a pressure support system configured to provide pressure support to the airway of a subject.

Obstructive sleep apnea (OSA) is a condition in which there is a decrease and/or cessation of airflow through an upper airway of a subject during sleep. Obstructive sleep apnea is the result of the upper airway collapsing and obstructing the airflow though the upper airway. The most common method of treatment for this condition is continuous positive airway pressure (CPAP). Other methods to treat sleep apnea using a positive airway pressure (PAP) device include Auto CPAP (APAP), bi-level positive airway pressure support (BiPAP), and servo ventilation. OSA may also be treated with drugs, surgeries, and/or other medical devices.

Accordingly, one or more aspects of the present disclosure relate to a pressure generation system for a pressure support system. The pressure generation system comprises a pressure generator, one or more sensors, and one or more processors. The pressure generator is configured to generate a pressurized flow of breathable gas for delivery to the airway of a subject. The one or more sensors are configured to generate output signals conveying information related to one or more parameters of the pressurized flow of breathable gas. The one or more processors are configured to execute computer program modules. The computer program modules comprise a respiratory event detection module, a pulse parameter module, and a control module. The respiratory event detection module is configured to detect airway obstruction. Airway obstruction is detected based on the output signals of the sensors. The pulse parameter module is configured to obtain one or more pulse parameters of a pulse of breathable gas. The pulse comprises a temporary bolus of breathable gas. The pulse is configured to open the airway of the subject. The control module is configured to control the pressure generator to generate, responsive to detection of airway obstruction, the pulse of breathable gas for delivery to the airway of the subject in accordance with the obtained pulse parameters.

Yet another aspect of the present disclosure relates to a method of generating pressure with a pressure generation system for a pressure support system. The pressure generation system comprises a pressure generator, one or more sensors, and one or more processors. The one or more processors are configured to execute computer program modules. The computer program modules comprise a respiratory event detection module, a pulse parameter module, and a control module. The method comprises generating a pressurized flow of breathable gas for delivery to the airway of a subject with the pressure generator; generating output signals conveying information related to one or more parameters of the pressurized flow of breathable gas with the one or more sensors; detecting airway obstruction with the airway obstruction module, wherein airway obstruction is detected based on the output signals generated by the sensors; obtaining one or more pulse parameters of a pulse of breathable gas with the pulse parameter module, the pulse comprising a temporary bolus of breathable gas, the pulse configured to open the airway of the subject; and controlling the pressure generator with the control module to generate, responsive to detection of airway obstruction, the pulse of breathable gas for delivery to the airway of the subject in accordance with the obtained pulse parameters.

Still another aspect of present disclosure relates to a pressure generation system for a pressure support system. The pressure generation system comprises means for generating a pressurized flow of breathable gas for delivery to the airway of a subject; means for generating output signals conveying information related to one or more parameters of the pressurized flow of breathable gas; and means for executing computer program modules. The computer program modules comprise means for detecting airway obstruction, wherein airway obstruction is detected based on the output signals; means for obtaining one or more pulse parameters of a pulse of breathable gas, the pulse comprising a temporary bolus of breathable gas, the pulse configured to open the airway of the subject; and means for controlling the means for generating a pressurized flow to generate, responsive to detection of airway obstruction, the pulse of breathable gas for delivery to the airway of the subject in accordance with the obtained pulse parameters.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

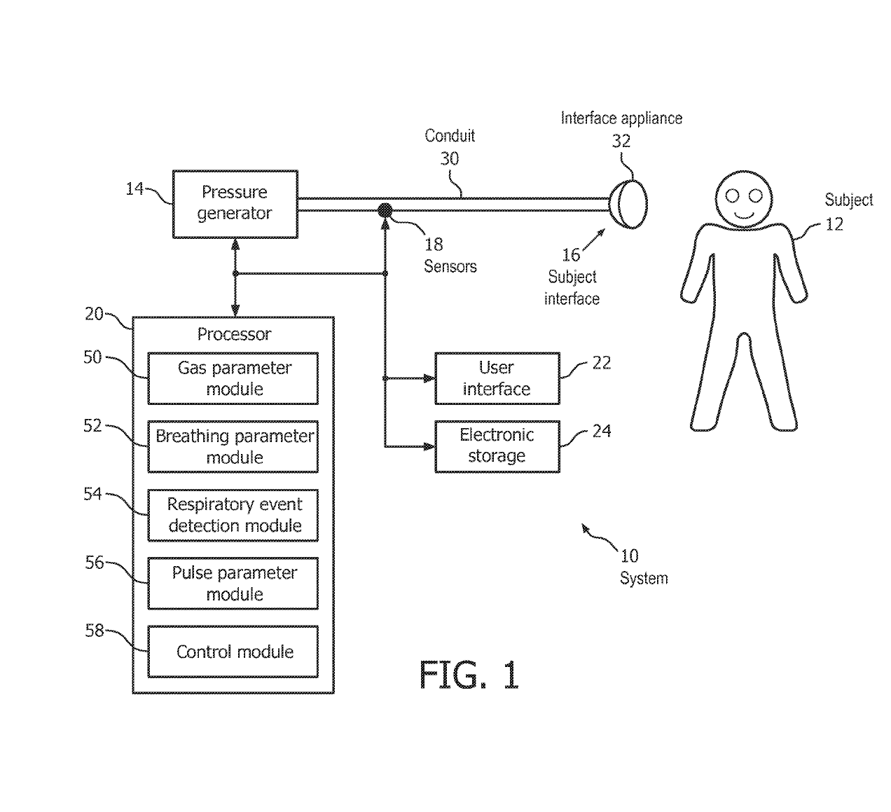

FIG. 1 is a schematic illustration of a pressure support system configured to provide pressure support to the airway of a subject.

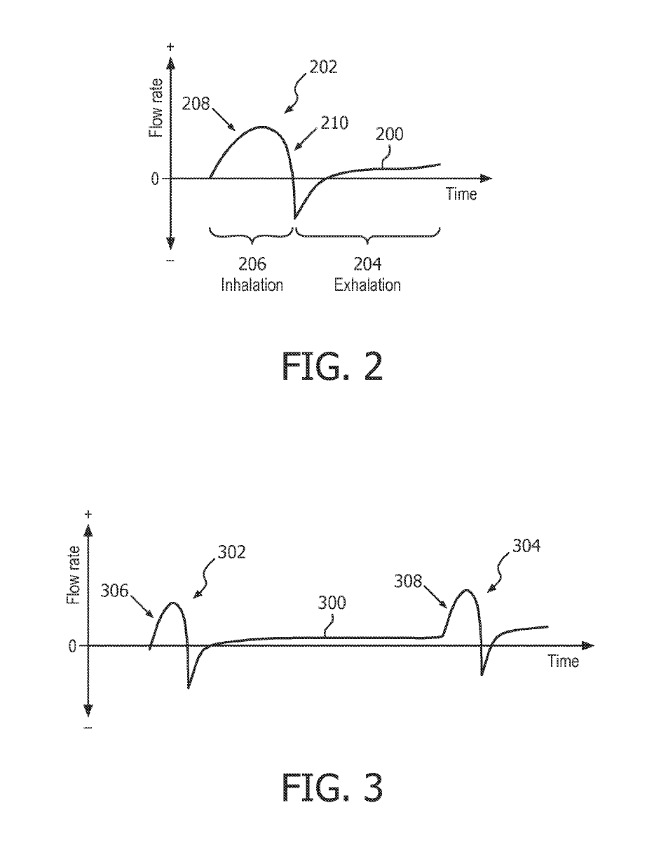

FIG. 2 shows a flow rate profile during a full breath.

FIG. 3 illustrates an occurrence of a respiratory event between two normal breaths.

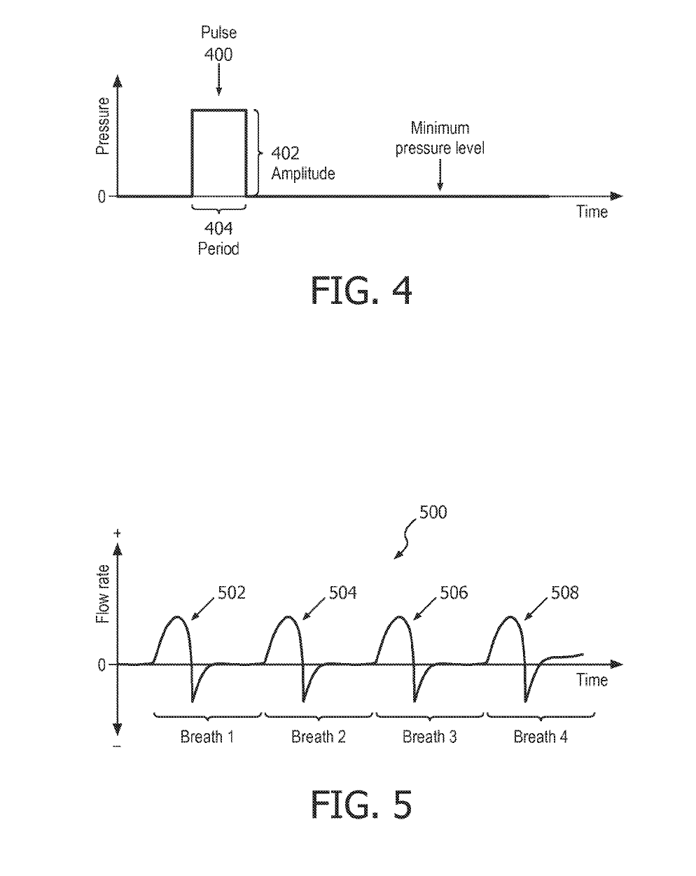

FIG. 4 illustrates a pressure pulse with an amplitude and a period.

FIG. 5 illustrates a regular flow rate profile for four consecutive breaths.

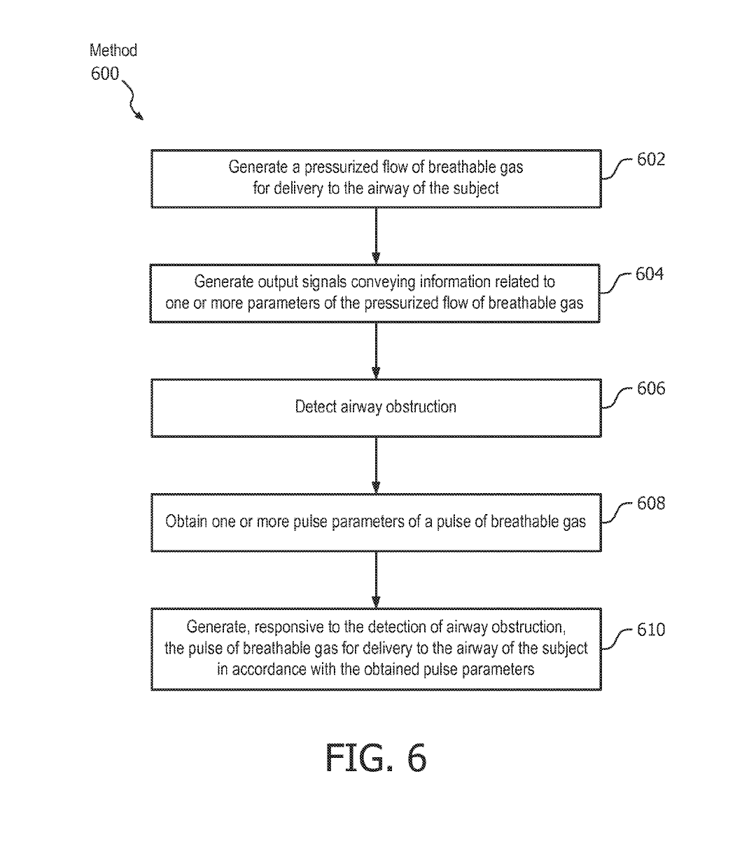

FIG. 6 illustrates a method of providing pressure support to the airway of a subject.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

FIG. 1 is a schematic illustration of a pressure support system 10 configured to provide pressure support to the airway of a subject 12. System 10 is configured to detect respiratory events and control the pressure support to generate a pulse of breathable gas for delivery to the airway of subject 12. The respiratory events may include airway obstruction (e.g., obstructive sleep apnea), and/or other respiratory events. The pulse may comprise a temporary bolus of breathable gas. The pulse may be delivered by system 10 as needed to open the airway of subject 12. The pulse of breathable gas is configured to open the airway of subject 12 before a subsequent inhalation.

Current positive airway pressure (e.g., CPAP, APAP, BiPAP) solutions for respiratory events such as obstructive sleep apnea may provide more pressure support than necessary to maintain an open airway. An airway may collapse at specific moments during sleep. Many times, it may only be necessary provide increased pressure to open the airway at the beginning of inhalation. Once air is flowing through the airway, airway collapse is less likely. There may be little or no need to maintain the same level of pressure after the beginning of inhalation. At the end of exhalation, pressure support provided by a typical PAP system may go unused by because there may be no inflow and/or outflow of air.

For example, FIG. 2 shows a flow rate profile 202 during a full breath. The flow rate increases 208 during inhalation 206, and then reverses direction 210 during exhalation 204. The relatively flat portion 200 of flow rate profile 202 at the end of exhalation 204 shows the lack of airflow at the end of exhalation 204.

Returning to FIG. 1, in some embodiments, system 10 comprises one or more of a pressure generator 14, a subject interface 16, one or more sensors 18, a processor 20, a user interface 22, electronic storage 24, and/or other components.

Pressure generator 14 is configured to generate a pressurized flow of breathable gas for delivery to the airway of subject 12. Pressure generator 14 may control one or more parameters of the flow of gas (e.g., flow rate, pressure, volume, temperature, duration, a timing, gas composition, etc.) for therapeutic purposes, and/or for other purposes. By way of a non-limiting example, pressure generator 14 may be configured to generate the pulse of breathable gas for delivery to the airway of subject 12. The pulse may have associated amplitude, period, timing, and/or other parameters.

Pressure generator 14 receives a flow of gas from a gas source, such as the ambient atmosphere, and elevates and/or reduces the pressure of that gas for delivery to the airway of a patient. Pressure generator 14 is any device, such as, for example, a pump, blower, piston, or bellows, that is capable of elevating and/or reducing the pressure of the received gas for delivery to a patient. Pressure generator 14 may comprise one or more valves for controlling the pressure and/or flow of gas, for example. The present disclosure also contemplates controlling the operating speed of the blower, either alone or in combination with such valves, to control the pressure and/or flow of gas provided to the patient.

Subject interface 16 is configured to deliver the pressurized flow of breathable gas to the airway of subject 12. As such, subject interface 16 comprises conduit 30, interface appliance 32, and/or other components. Conduit 30 is configured to convey the pressurized flow of gas to interface appliance 32. Conduit 30 may be a flexible length of hose, or other conduit, that places interface appliance 32 in fluid communication with pressure generator 14. Interface appliance 32 is configured to deliver the flow of gas to the airway of subject 12. In some embodiments, interface appliance 32 is non-invasive. As such, interface appliance 32 non-invasively engages subject 12. Non-invasive engagement comprises removably engaging an area (or areas) surrounding one or more external orifices of the airway of subject 12 (e.g., nostrils and/or mouth) to communicate gas between the airway of subject 12 and interface appliance 32. Some examples of non-invasive interface appliance 32 may comprise, for example, a nasal cannula, a nasal mask, a nasal/oral mask, a full face mask, a total face mask, or other interface appliances that communicate a flow of gas with an airway of a subject. The present disclosure is not limited to these examples, and contemplates delivery of the flow of gas to the subject using any interface appliance, including an invasive interface appliance such as an endotracheal tube and/or other appliances.

Sensors 18 are configured to generate output signals conveying information related to one or more gas parameters of the pressurized flow of breathable gas. Sensors 18 are configured to generate output signals conveying information related to one or more breathing parameters related to the respiration of subject 12. The one or more gas parameters and/or the one or more breathing parameters may comprise one or more of a flow rate, a volume, a pressure, a composition (e.g., concentration(s) of one or more constituents), temperature, humidity, acceleration, velocity, acoustics, changes in a parameter indicative of respiratory effort by subject 12, a timing, a duration, a frequency, and/or other parameters. Sensors 18 may comprise one or more sensors that measure such parameters directly (e.g., through fluid communication with the flow of gas in subject interface 16). Sensors 18 may comprise one or more sensors that generate output signals related to one or more parameters of the flow of gas indirectly. For example, one or more of sensors 18 may generate an output based on an operating parameter of pressure generator 14 (e.g., a valve driver or motor current, voltage, rotational velocity, and/or other operating parameters). Although sensors 18 are illustrated at a single location within (or in communication with) conduit 30 between interface appliance 32 and pressure generator 14, this is not intended to be limiting. Sensors 18 may include sensors disposed in a plurality of locations, such as for example, within pressure generator 14, within (or in communication with) interface appliance 32, in communication with subject 12, and/or in other locations.

Processor 20 is configured to provide information processing capabilities in system 10. As such, processor 20 may comprise one or more of a digital processor, an analog processor, and a digital circuit designed to process information, an analog circuit designed to process information, a state machine, and/or other mechanisms for electronically processing information. Although processor 20 is shown in FIG. 1 as a single entity, this is for illustrative purposes only. In some implementations, processor 20 may comprise a plurality of processing units. These processing units may be physically located within the same device (e.g., pressure generator 14), or processor 20 may represent processing functionality of a plurality of devices operating in coordination.

As shown in FIG. 1, processor 20 is configured to execute one or more computer program modules. The one or more computer program modules may comprise one or more of a gas parameter module 50, a breathing parameter module 52, a respiratory event detection module 54, a pulse parameter module 56, a control module 58, and/or other modules. Processor 20 may be configured to execute modules 50, 52, 54, 56, and/or 58 by software; hardware; firmware; some combination of software, hardware, and/or firmware; and/or other mechanisms for configuring processing capabilities on processor 20.

It should be appreciated that although modules 50, 52, 54, 56, and 58 are illustrated in FIG. 1 as being co-located within a single processing unit, in implementations in which processor 20 comprises multiple processing units, one or more of modules 50, 52, 54, 56, and/or 58 may be located remotely from the other modules. The description of the functionality provided by the different modules 50, 52, 54, 56, and/or 58 described below is for illustrative purposes, and is not intended to be limiting, as any of modules 50, 52, 54, 56, and/or 58 may provide more or less functionality than is described. For example, one or more of modules 50, 52, 54, 56, and/or 58 may be eliminated, and some or all of its functionality may be provided by other modules 50, 52, 54, 56, and/or 58. As another example, processor 20 may be configured to execute one or more additional modules that may perform some or all of the functionality attributed below to one of modules 50, 52, 54, 56, and/or 58.

Gas parameter module 50 is configured to determine one or more gas parameters of the pressurized flow of breathable gas. Gas parameter module 50 is configured to determine the one or more gas parameters based on the output signals of sensors 18. The one or more gas parameters of the pressurized flow of breathable gas may comprise, for example, one or more of a flow rate, a volume, a pressure, humidity, temperature, acceleration, velocity, and/or other gas parameters. The information determined by gas parameter module 50 may be used for controlling pressure generator 14, determining breathing parameters of subject 12, and/or other uses.

Breathing parameter module 52 is configured to determine one or more breathing parameters of subject 12. The one or more breathing parameters are determined based on the output signals of sensors 18, information determined by gas parameter module 50, and/or based on other information. The breathing parameters may indicate a respiratory effort of subject 12. This includes one or more of a thoracic respiratory effort, an abdominal respiratory effort, and/or other parameters indicating respiratory effort. The one or more breathing parameters may include, for example, a tidal volume, a composition, a timing (e.g., beginning and/or end of inhalation, beginning and/or end of exhalation, etc.), a duration (e.g., of inhalation, of exhalation, of a single breathing cycle, etc.), a breath rate, a respiration frequency, and/or other parameters.

In some embodiments, breathing parameter module 52 is configured to determine one or more baseline levels of the one or more breathing parameters. The one or more baseline levels of the one or more breathing parameters may be related to normal respiration of subject 12. In some embodiments, breathing parameter module 52 determines the one or more baseline levels of the one or more breathing parameters based on previous respiration by subject 12. By way of a non-limiting example, breathing parameter module 52 may determine at least one baseline level of at least one breathing parameter for each inhalation in a series of consecutive inhalations. The at least one determined breathing parameter may include, for instance, a tidal volume, and/or other breathing parameters. Breathing parameter module 52 may determine a baseline tidal volume level for each inhalation. By way of another non-limiting example, breathing parameter module 52 may determine at least one breathing parameter for a series of consecutive inhalations in addition to the breathing parameter determined for each individual inhalation. For example, breathing parameter module 52 may determine an average tidal volume for a series of consecutive inhalations. Breathing parameter module 52 may determine an average baseline tidal volume level for the series of consecutive inhalations.

Respiratory event detection module 54 is configured to detect respiratory sleep events. These events include events that result in irregular breathing during sleep or near sleep states. Such events may include, for example, airway obstruction and/or other events. Respiratory sleep events including airway obstruction may be detected based on the output signals of sensors 18, information determined by gas parameter module 50, information determined by breathing parameter module 52, and/or based on other information. In some embodiments, respiratory sleep events detected by respiratory event detection module 54 may be related to obstructive sleep apnea.

Respiratory event detection module 54 is configured to detect respiratory sleep events (e.g., airway obstruction) during normal breathing of subject 12. In some embodiments, the information indicating respiratory sleep events comprises one or more gas parameters (e.g. pressure, flow rate, and/or other gas parameters), one or more breathing parameters (e.g., a tidal volume, a composition, a timing, a duration, a breath rate, peak flow, airway pressure, and/or other breathing parameters), and/or other information. Detection of respiratory sleep events may comprise, for example, output signals and/or parameters that indicate respiratory effort (e.g., thoracic respiratory effort and/or abdominal respiratory effort) without a corresponding inflow of breathable gas, a reduction in tidal volume, and/or changes in other output signals and/or parameters. In some embodiments, respiratory sleep events may be determined based on a comparison of parameters from a current inhalation to parameters determined during previous respiration by subject 12. In some embodiments, respiratory event detection module 54 may be configured to detect respiratory sleep events such as airway obstruction based on forced oscillation technique. This is not intended to be limiting, as any number of techniques for detecting respiratory sleep events could be implemented without departing from the scope of this disclosure.

For example, FIG. 3 illustrates an occurrence of an event 300 between two normal breaths 302, 304. In some embodiments, event 300 may indicate obstructive sleep apnea, for example. In the example shown in FIG. 3, event 300 may be detected because the flow rate did not increase 306, 308 at event 300 as it would during a normal inhalation.

Returning to FIG. 1, in some embodiments, respiratory event detection module 54 is configured to detect respiratory sleep events based on one or more gas parameters, one or more breathing parameters, and/or one or more other parameters breaching a threshold level (e.g., pressure and/or flow rate breaching threshold(s)). The threshold levels may be configurable to a user (e.g., subject 12, a doctor, a caregiver, a researcher, and/or other users), predefined at manufacture, determined based on previous respiration by subject 12, and/or determined in other manners. The threshold levels may include the one or more baseline levels of the one or more breathing parameters determined by breathing parameter module 52. For example, airway obstruction may be detected responsive to subject 12 not reaching a minimum inhaled tidal volume in a given amount of time for a current breath after exhalation of a previous breath.

Pulse parameter module 56 is configured to obtain one or more pulse parameters of the pulse of breathable gas. The pulse of breathable gas may comprise a temporary bolus of breathable gas. The pulse of breathable gas is configured to open the airway of subject 12 prior to a subsequent inhalation. The one or more pulse parameters for the pulse are obtained before generation of the pulse. The parameters may be obtained based on statistical analysis of the effectiveness of previous pulses of breathable gas. The set of initial pulse parameters could be set from a menu with multiple parameter options. However, once enough data is collected by processor 20, the set of initial conditions may be dynamically adjusted during each usage as more information on the effectiveness of the pulses is gathered by processor 20. The pulse parameters may include, for example, one or more of amplitude, a period, a timing of the pulse, and/or other parameters.

For example, FIG. 4 illustrates a pressure pulse 400 with amplitude 402 and a period 404. In some embodiments, a form of the pressure pulse (e.g., a waveform) may be configured as a square wave (as shown in FIG. 4), a sine wave, a triangular wave, and/or other wave form shapes.

Returning to FIG. 1, pulse parameter module 56 may be configured to adjust the pulse parameters of the pulse of breathable gas. The adjustment of the pulse parameters is configured to enhance an effectiveness of a current pulse relative to the effectiveness of one or more previous pulses. The effectiveness of a pulse is related to and/or quantified by an amount the airway of subject 12 is opened, the extent to which subject 12 begins breathing, and/or other information. The adjustment is based on information conveyed by the output signals from sensors 18 during the one or more previous pulses, information determined by gas parameter module 50, information determined by breathing parameter module 52, information entered by subject 12 and/or other users to user interface 22, and/or other information. In some embodiments, pulse parameter module 56 may be configured to determine the effectiveness of a pulse by analyzing gas and/or breathing parameter information related to one or more previous pulses and the one or more corresponding inhalations. The analyzed information may be used to adjust the current pulse. The adjustment comprises changing one or more of the amplitude (e.g., the pressure level of the pulse), the period, the timing, and/or other parameters of the pulse. In some embodiments, the period (e.g., the duration) is kept to a minimum level while the amplitude, timing, and/or other parameters are adjusted. In some embodiments, the shape of the pressure pulse waveform may be adjusted by adjusting the amplitude, period, timing, and/or other parameters of the pulse.

By way of a first non-limiting example, if a given pressure pulse had a pressure support level of 3 cmH₂O and was ineffective in opening the airway of subject 12, the next pressure pulse may be 4 cmH₂O. By way of a second non-limiting example, another adjustment may increase and/or decrease the period (duration) of the pressure pulse. In some embodiments, the period of the pressure pulse may be kept shorter than the inhalation time. In some embodiments, the period of the pressure pulse may be less than approximately half of inhalation time such that the pulse is a temporary bolus of breathable gas.

By way of a third non-limiting example, pulse parameter module 56 may be configured to adjust a timing of the pulse of breathable gas. The timing of the pulse of breathable gas may be based on a breathing rate calculation. If the breathing rate of subject 12 is ten breaths per minute, subject 12 has six second breaths on average. Thus, if subject 12 does not inhale normally, a pulse may be generated at most after six seconds. Assuming a one half inhalation time to exhalation time ratio for this example, an optimal delivery of the pressure pulse may be four seconds after the end of the previous inhalation. However, a pressure pulse may be delivered every six seconds to avoid user arousals. Control module 58 may be configured such that if an exhalation last longer than four seconds (e.g., if subject 12 does not inhale normally) and/or a respiratory sleep event (e.g., airway obstruction) is detected, a pressure pulse may be delivered. It should be noted that the assumptions and/or timings discussed in this example related to the timing of a pulse are not intended to be limiting.

Control module 58 is configured to control pressure generator 14 to generate the pulse of breathable gas for delivery to the airway of subject 12. Control module 58 is configured to control pressure generator 14 to deliver the pulse responsive to detection of respiratory sleep events (e.g., airway obstruction) by respiratory event detection module 54. The pulse is delivered to the airway of subject 12 such that the airway of subject 12 is opened prior to a subsequent inhalation and/or ventilation. Control module 58 is configured to control pressure generator 14 to deliver the pulse in accordance with the pulse parameters obtained and/or adjusted by pulse parameter module 56 prior to the pulse. For example, control module 58 may be configured to control pressure generator 14 to deliver the pulse responsive to detection of airway obstruction and/or in accordance with a timing obtained by pulse parameter module 56. In some embodiments, the pulse of breathable gas can be a square wave. Moreover, the square wave can have smooth edges to increase comfort and decrease the likelihood of arousing subject 12. In some embodiments, the pulse of breathable gas can be a saw wave with a ramping time adjusted based on efficacy of the therapy to mitigate obstructions during respiration. Finally, the parameters that will control the duration and magnitude of the pulse of breathable gas may initially be selected from a control menu and then automatically adjusted by control module 58 based on the statistical effectiveness in clearing the airway obstruction of the previous pulses.

Control module 58 is configured to control pressure generator 14 to provide the pressurized flow of breathable gas to the airway of the subject according to a positive airway pressure support therapy regime (e.g., CPAP, APAP, BiPAP). Control module 58 is configured to control pressure generator 14 to provide a minimum amount of positive airway pressure support during inhalation (e.g., IPAP) and/or exhalation (e.g., EPAP). Delivering a minimum amount of pressure support and providing a pulse when needed may increase the comfort level of subject 12 during therapy. The minimum amount of positive airway pressure support may comprise delivering the pressurized flow of breathable gas at a minimum pressure level. The minimum pressure level may be configured such that carbon dioxide re-breathing is substantially avoided during pressure support therapy. In some embodiments, the inhalation positive airway pressure may be less than about 7mmH₂O. In some embodiments, the inhalation positive airway pressure may be between about 1 cmH₂O and about 7cmH₂O. In some embodiments, the inhalation positive airway pressure level may be zero if there is no carbon dioxide rebreathing by subject 12. Control module 58 controls pressure generator 14 to deliver the pulse as needed in addition to the positive airway pressure support.

Control module 58 is configured to control pressure generator 14 to generate pulses as needed such that subject 12 may maintain a regular breathing pattern. FIG. 5 illustrates a regular flow rate profile 500 for four consecutive breaths 502, 504, 506, and 508. One or more of breaths 502, 504, 506, and/or 508 may be preceded by the control module (shown in FIG. 1) controlling the pressure generator (shown in FIG. 1) to generate a pulse for delivery to the airway of the subject (shown in FIG. 1) such that the subject's airway is opened prior to and/or at the beginning of the inhalation.

Returning to FIG. 1, user interface 22 is configured to provide an interface between system 10 and subject 12 and/or other users through which subject 12 and/or other users may provide information to and receive information from system 10. User interface 22 may be configured to receive entry and/or selection of control inputs related to the positive airway pressure support therapy regime, the pulse parameters, and/or other information from subject 12 and/or other users. Other users may comprise a caregiver, a doctor, a decision maker, and/or other users. This enables data, cues, results, and/or instructions and any other communicable items, collectively referred to as “information,” to be communicated between a user (e.g., subject 12) and one or more of pressure generator 14, processor 20, and/or other components of system 10. Examples of interface devices suitable for inclusion in user interface 22 comprise a keypad, buttons, switches, a keyboard, knobs, levers, a display screen, a touch screen, speakers, a microphone, an indicator light, an audible alarm, a printer, a tactile feedback device, and/or other interface devices. In some embodiments, user interface 22 comprises a plurality of separate interfaces. In some embodiments, user interface 22 comprises at least one interface that is provided integrally with pressure generator 14.

It is to be understood that other communication techniques, either hard-wired or wireless, are also contemplated by the present disclosure as user interface 22. For example, the present disclosure contemplates that user interface 22 may be integrated with a removable storage interface provided by electronic storage 24. In this example, information may be loaded into system 10 from removable storage (e.g., a smart card, a flash drive, a removable disk, etc.) that enables the user(s) to customize the implementation of system 10. Other exemplary input devices and techniques adapted for use with system 10 as user interface 22 comprise, but are not limited to, an RS-232 port, RF link, an IR link, modem (telephone, cable or other). In short, any technique for communicating information with system 10 is contemplated by the present disclosure as user interface 22.

In some embodiments, electronic storage 24 comprises electronic storage media that electronically stores information. The electronic storage media of electronic storage 24 may comprise one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 24 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 24 may store software algorithms, information determined by processor 20, information received via user interface 22, and/or other information that enables system 10 to function properly. Electronic storage 24 may be (in whole or in part) a separate component within system 10, or electronic storage 24 may be provided (in whole or in part) integrally with one or more other components of system 10 (e.g., user interface 22, processor 20, etc.).

FIG. 6 illustrates a method 600 for providing pressure support to the airway of a subject with a pressure support system. The system comprises a pressure generator, one or more sensors, and one or more processors. The one or more processors are configured to execute computer program modules. The computer program modules comprise a respiratory event detection module, a pulse parameter module, and a control module. The operations of method 600 presented below are intended to be illustrative. In some embodiments, method 600 may be accomplished with one or more additional operations not described, and/or without one or more of the operations discussed. Additionally, the order in which the operations of method 600 are illustrated in FIG. 6 and described below is not intended to be limiting.

In some embodiments, method 600 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 600 in response to instructions stored electronically on an electronic storage medium. The one or more processing devices may include one or more devices configured through hardware, firmware, and/or software to be specifically designed for execution of one or more of the operations of method 600.

At an operation 602, a pressurized flow of breathable gas is generated for delivery to an airway of a subject. In some embodiments, operation 602 is performed by a pressure generator the same as or similar to pressure generator 14 (shown in FIG. 1 and described herein).

At an operation 604, output signals conveying information related to one or more parameters of the pressurized flow of breathable gas are generated. In some embodiments, operation 604 is performed by sensors the same as or similar to sensors 18 (shown in FIG. 1 and described herein).

At an operation 606, airway obstruction is detected. At operation 606, other respiratory sleep events instead of and/or in addition to airway obstruction may be detected. In some embodiments, operation 606 is performed by a computer program module the same as or similar to respiratory event detection module 54 (shown in FIG. 1 and described herein).

At an operation 608, one or more pulse parameters of a pulse of breathable gas are obtained. In some embodiments, operation 608 is performed by a computer program module the same as or similar to pulse parameter module 56 (shown in FIG. 1 and described herein).

At an operation 610, responsive to the detection of airway obstruction and/or other respiratory sleep events, the pulse of breathable gas for is generated for delivery to the airway of the subject in accordance with the obtained pulse parameters. In some embodiments, operation 610 is performed by a pressure generator the same as or similar to pressure generator 14 (shown in FIG. 1 and described herein). In some embodiments, the pressure generator is controlled to generate the pulse of breathable gas by a computer program module the same as or similar to control module 58 (shown in FIG. 1 and described herein).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

I/We claim:

1. A pressure generation system for a pressure support system the pressure generation system comprising:
a pressure generator configured to generate a pressurized flow of breathable gas for delivery to the airway of a subject;
one or more sensor configured to generate output signals conveying information related to one or more parameters of the pressurized flow of breathable gas; and
one or more processors configured to execute computer program modules, the computer program modules comprising:
a respiratory event detection module configured to detect airway obstruction, wherein airway obstruction is detected based on the output signals of the sensors;
a pulse parameter module configured to obtain one or more pulse parameters of a pulse of breathable gas, the pulse comprising a temporary bolus of breathable gas, the pulse configured to open the airway of the subject, wherein the pulse parameters of the pulse of breathable gas are obtained before generation of the pulse and define the pulse from beginning to end, wherein the pulse parameters include an amplitude of a first pulse parameter, a period of the first pulse parameter, and/or a timing of the first pulse parameter; and
a control module configured to control the pressure generator to generate, responsive to detection of airway obstruction, the pulse of breathable gas for delivery to the airway of the subject in accordance with the obtained pulse parameters.

2. (canceled)

3. The system of claim 1, wherein the computer program modules further comprise a breathing parameter module configured to determine one or more baseline levels of one or more breathing parameters of the subject, the one or more baseline levels of the one or more breathing parameters related to normal respiration of the subject, the one or more baseline levels of the one or more breathing parameters determined based on the output signals of the sensors,
and wherein the respiratory event detection module is configured to detect airway obstruction responsive to one or more of the breathing parameters breaching one or more of the baseline levels.

4. The system of claim 1, wherein the pulse parameter module is configured to adjust the pulse parameters of the pulse of breathable gas, the adjustment configured to enhance an effectiveness of a current pulse relative to one or more previous pulses, the effectiveness related to an amount the airway of the subject is opened, the adjustment based on information conveyed by the output signals during the one or more previous pulses, the adjustment comprising changing one or more of the amplitude of the first pulse parameter, the period of the first pulse parameter, or the timing of the first pulse parameter.

5. The system of claim 1, wherein the control module is configured to control the pressure generator to provide the pressurized flow of breathable gas to the airway of the subject according to a positive airway pressure support therapy regime, wherein the control module is configured to control the pressure generator to provide a minimum amount of positive pressure airway support, and wherein the control module controls the pressure generator to deliver the pulse in addition to the positive airway pressure support.

6. A method for generating pressure with a pressure generation system for a pressure support system, the pressure generation system comprising a pressure generator, one or more sensors, and one or more processors, the one or more processors configured to execute computer program modules, the computer program modules comprising an respiratory event detection module, a pulse parameter module, and a control module, the method comprising:
generating a pressurized flow of breathable gas for delivery to the airway of a subject with the pressure generator;
generating output signals conveying information related to one or more parameters of the pressurized flow of breathable gas with the one or more sensors;
detecting airway obstruction with the airway obstruction module, wherein airway obstruction is detected based on the output signals generated by the sensors;
obtaining one or more pulse parameters of a pulse of breathable gas with the pulse parameter module, the pulse comprising a temporary bolus of breathable gas, the pulse configured to open the airway of the subject, wherein the pulse parameters of the pulse of breathable gas are obtained before generation of the pulse and define the pulse from beginning to end, wherein the pulse parameters include an amplitude of a first pulse parameter, a period of the first pulse parameter, and/or a timing of the first pulse parameter; and
controlling the pressure generator with the control module to generate, responsive to detection of airway obstruction, the pulse of breathable gas for delivery to the airway of the subject in accordance with the obtained pulse parameters.

7. (canceled)

8. The method of claim 6, wherein the computer program modules further comprise a breathing parameter module, the method further comprising determining one or more baseline levels of one or more breathing parameters of the subject with the breathing parameter module, the one or more baseline levels of the one or more breathing parameters related to normal respiration of the subject, the one or more baseline levels of the one or more breathing parameters determined based on the output signals generated by the sensors,
and wherein airway obstruction is detected responsive to one or more of the breathing parameters breaching one or more of the baseline levels.

9. The method of claim 6, further comprising adjusting the pulse parameters of the pulse of breathable gas with the pulse parameter module, the adjustment configured to enhance an effectiveness of a current pulse relative to one or more previous pulses, the effectiveness related to an amount the airway of the subject is opened, the adjustment based on information conveyed by the output signals during the one or more previous pulses, the adjustment comprising changing one or more of the amplitude of the first pulse parameter, the period of the first pulse parameter, or the timing of the first pulse parameter.

10. The method of claim 6, further comprising controlling the pressure generator with the control module to provide the pressurized flow of breathable gas to the airway of the subject according to a positive airway pressure support therapy regime, controlling the pressure generator to provide a minimum amount of positive pressure airway support, and controlling the pressure generator to deliver the pulse in addition to the positive airway pressure support.

11. A pressure generation system for a pressure support system, the pressure generation system comprising:
means for generating a pressurized flow of breathable gas for delivery to the airway of a subject;
means for generating output signals conveying information related to one or more parameters of the pressurized flow of breathable gas; and
means for executing computer program modules, the computer program modules comprising:
means for detecting airway obstruction, wherein airway obstruction is detected based on the output signals;
means for obtaining one or more pulse parameters of a pulse of breathable gas, the pulse comprising a temporary bolus of breathable gas, the pulse configured to open the airway of the subject, wherein the pulse parameters of the pulse of breathable gas are obtained before generation of the pulse and define the pulse from beginning to end, wherein the pulse parameters include an amplitude of a first pulse parameter, a period of the first pulse parameter, and/or a timing of the first pulse parameter; and
means for controlling the means for generating a pressurized flow to generate, responsive to detection of airway obstruction, the pulse of breathable gas for delivery to the airway of the subject in accordance with the obtained pulse parameters.

12. (canceled)

13. The system of claim 11, wherein the computer program modules further comprise means for determining one or more baseline levels of one or more breathing parameters of the subject, the one or more baseline levels of the one or more breathing parameters related to normal respiration of the subject, the one or more baseline levels of the one or more breathing parameters determined based on the output signals of the sensors,
and wherein the means for detecting airway obstruction is configured to detect airway obstruction responsive to one or more of the breathing parameters breaching one or more of the baseline levels.

14. The system of claim 11, wherein the means for obtaining one or more pulse parameters is configured to adjust the pulse parameters of the pulse of breathable gas, the adjustment configured to enhance an effectiveness of a current pulse relative to one or more previous pulses, the effectiveness related to an amount the airway of the subject is opened, the adjustment based on information conveyed by the output signals during the one or more previous pulses, the adjustment comprising changing one or more of the amplitude of the first pulse parameter, the period of the first pulse parameter, or a timing of the first pulse parameter.

15. The system of claim 11, wherein the means for controlling is configured to control the means for generating the pressurized flow to provide the pressurized flow of breathable gas to the airway of the subject according to a positive airway pressure support therapy regime, wherein the means for controlling is configured to control the means for generating the pressurized flow to provide a minimum amount of positive pressure airway support, and wherein the means for controlling controls the means for generating the pressurized flow to deliver the pulse in addition to the positive airway pressure support.
